(19) United States
(12) Patent Application Publication
(10) Pub. No.: US 2025/0302765 A1
(43) Pub. Date: Oct. 2, 2025

(54) TRIACETYL ANDROGRAPHOLIDE NANOCRYSTAL AND PREPARATION METHOD AND APPLICATION THEREOF

(71) Applicant: Henan University, Zhengzhou (CN)

(72) Inventors: Guang HAN, Zhengzhou (CN); Junli ZHAO, Zhengzhou (CN); Yu ZHANG, Zhengzhou (CN); Chang REN, Zhengzhou (CN); Mengjun ZHANG, Zhengzhou (CN); Jiyao LI, Zhengzhou (CN); Kunlin ZHANG, Zhengzhou (CN); Zongzheng SUN, Zhengzhou (CN); Xin SUI, Zhengzhou (CN); Qing WEN, Zhengzhou (CN)

(73) Assignee: Henan University, Zhengzhou (CN)

(21) Appl. No.: 19/095,788

(22) Filed: Mar. 31, 2025

(30) Foreign Application Priority Data
Apr. 1, 2024 (CN) 202410385289.7

(51) Int. Cl. A61K 9/51; A61K 31/365; A61P 11/00

(52) U.S. Cl. CPC A61K 9/5123; A61K 9/5146; A61K 9/5192; A61K 31/365; A61P 11/00

(57) ABSTRACT

The triacetyl andrographolide nanocrystal is mainly composed of triacetyl andrographolide, a stabilizer and an excipient, and an average particle size of drug particles in a triacetyl andrographolide nanocrystal suspension obtained by redissolving the triacetyl andrographolide nanocrystal in water, is less than 500 nm, and a PDI is less than 0.2. The nanocrystal suspension is prepared from the triacetyl andrographolide and the stabilizer by a high-speed shear anti-solvent method in combination with a high-pressure homogenization method, then the excipient is added, and the nanocrystal is prepared through spray-drying.

### CROSS-REFERENCE TO RELATED APPLICATIONS

The present application claims priority to the Chinese patent application No. 202410385289.7 filed to the China National Intellectual Property Administration on Apr. 1, 2024 and entitled “TRIACETYL ANDROGRAPHOLIDE NANOCRYSTAL AND PREPARATION METHOD AND APPLICATION THEREOF”, the entire content of which is incorporated herein by reference.

### TECHNICAL FIELD

The present disclosure belongs to the technical field of medicines, and particularly relates to a triacetyl andrographolide nanocrystal and a preparation method and application thereof.

### BACKGROUND

Triacetyl andrographolide is a derivative obtained by performing esterifying modification on hydroxyl at positions 3, 14 and 19 of andrographolide, and has pharmacological actions such as anti-inflammation, anti-tumor, neuroprotection and immunomodulation (Chinese patents ZL200510017654.6, ZL200710148499.0 and ZL200710016490.4, and references Phytotherapy Research, 2023, 37 (2): 410-423, Pharmacological Research, 2019, 144:227-234, and Chinese Pharmaceutical Journal, 2017, 52 (9): 738-742, etc.). Research has found that the absolute bioavailability of the triacetyl andrographolide is 7.19%, which is more than 7 times higher than that of andrographolide. Its pharmaceutical effects on lung injury-related disease model mice are significantly improved. However, due to the extremely low water solubility of drugs, there are problems such as high dosage and poor druggability. Examples in the Chinese patent ZL200710016490.4 disclose preparation of an emulsion from triacetyl andrographolide to enhance its pharmaceutical effects, but only provide the preparation of the emulsion and an evaluation of its efficacy in reducing the contents of TNF-α and IL-1B in serums of rabbits with endotoxic shock, without providing data on improving drug dissolution, increasing bioavailability, or pharmacodynamic treatment for lung injury-related diseases such as acute lung injury and chronic obstructive pulmonary disease. Therefore, it is impossible to judge the therapeutic effect of the triacetyl andrographolide on lung injury and the impact of the emulsion prepared from the drug on the pharmaceutical effect of the lung injury-related diseases. At the same time, the emulsion has the problems such as large amounts of excipients, low drug loading, and poor stability of a preparation.

A nanocrystal technology is a preparation technology that directly processes drugs into nanoparticles. Due to the fact that drugs often retain their crystalline forms, they are commonly referred to as nanocrystal drugs (abbreviated as nanocrystals), the solubility of poorly soluble drugs and the dissolution rate of their preparations can be improved, and the bioavailability of the drugs can be increased. Meanwhile, compared with traditional nanocarrier-encapsulated drugs such as emulsions, preparation of the nanocrystals requires only a small amount of stabilizers, which has the advantages such as high drug loading, low cost, and easy production and scaling up of the process. At present, multiple nanocrystal drug preparations have been launched, such as Aprepitant nanocrystal capsules (Emend) and Megace ES nanocrystal suspensions. Multiple Chinese patents that use the nanocrystal technology to improve drug bioavailability have been disclosed (ZL202211356947.7, ZL202210550188.1, ZL202111598896.4, etc.).

Lung injury refers to lung diseases caused by microbial infections or stimulation of mechanical foreign objects such as cigarette smoke and haze particles in the respiratory tract and lungs, and includes acute lung injury, chronic obstructive pulmonary disease, etc., in which the chronic obstructive pulmonary disease is the most common. The chronic obstructive pulmonary disease is a chronic airway disease characterized by airway limitation. Airway limitation is not completely reversible and develops progressively, manifested as complex airway inflammation responses, oxidative stress, protease/antiprotease imbalance, etc. By 2020, the chronic obstructive pulmonary disease has become the third leading cause of death in the world and a major chronic disease “on the same scale” as hypertension and diabetes. According to the China Adult Pulmonary Health Report in 2018, the number of chronic obstructive pulmonary disease patients in China is close to 100 million, with 1 million deaths per year. At present, a main strategy for clinical treatment of the chronic obstructive pulmonary disease is combination therapy of bronchodilators and glucocorticoids. However, long-term use of the glucocorticoids such as dexamethasone and budesonide has the side effects of suppressing the body's immune functions, leading to osteoporosis, etc. The development of novel chronic obstructive pulmonary disease treatment drugs has become a research hotspot.

The Chinese patent ZL200710016490.4 discloses triacetyl andrographolide as an inhibitor of TNF-α and IL-1B for the treatment of the chronic obstructive pulmonary disease. However, in examples, it is only reported that the triacetyl andrographolide can reduce the contents of TNF-α and IL-1β in a supernatant of an LPS-induced RAW264.7 cell culture fluid and in serums of rabbits with endotoxic shock, and there is a lack of in vivo and in vitro pharmacodynamic data for the treatment of the chronic obstructive pulmonary disease. In addition, as a chronic airway disease characterized by airway limitation, the improvement of lung functions is the key to evaluating the drug treatment effect for the chronic obstructive pulmonary disease. This patent only involves the anti-inflammatory effect of drugs and lacks evaluation of the effects of the drugs on a pulmonary ventilation function (0.1 s forced expiratory volume (FEV_{0.1}), forced vital capacity (FVC), 0.1 second rate (FEV_{0.1}/FVC), total lung capacity (TLC), functional residual capacity (FRC), airway resistance (RI), dynamic lung compliance (Cydn), etc.), alveolar and airway wall structures, protease levels, and other indicators in chronic obstructive pulmonary disease model animals. Therefore, it is impossible to judge the therapeutic effect of the triacetyl andrographolide on the chronic obstructive pulmonary disease.

### SUMMARY OF THE INVENTION

For the problems of low water solubility, high drug use dosage and poor druggability of triacetyl andrographolide, the present disclosure proposes a triacetyl andrographolide nanocrystal and a preparation method and application thereof, which can effectively increase drug absorption and bioavailability, improve the effects of treatment of lung injury-related diseases, lower the drug use dosage, and improve the druggability, thereby facilitating preparation processing and application.

In order to implement the above objectives, a technical solution of the present disclosure is implemented as follows.

A triacetyl andrographolide nanocrystal is provided. The triacetyl andrographolide nanocrystal is mainly composed of triacetyl andrographolide, a stabilizer and an excipient, and an average particle size of drug particles in a triacetyl andrographolide nanocrystal suspension obtained by redissolving the triacetyl andrographolide nanocrystal in water, is less than 500 nm, and a PDI is less than 0.2.

The triacetyl andrographolide nanocrystal has the same crystal form as a triacetyl andrographolide active pharmaceutical ingredient, and both of them exhibit obvious crystalline diffraction peaks in their XRD spectra at 2θ values of 11.71°, 11.83°, 12.38°, 12.50°, 13.46°, 14.37°, 19.63° and 20.48°.

A triacetyl andrographolide nanocrystal preparation includes the above triacetyl andrographolide nanocrystal and conventional pharmaceutic adjuvants in the art.

The triacetyl andrographolide nanocrystal preparation includes a solid preparation, a liquid preparation and a semi-solid preparation, and administration routes include oral administration, pulmonary inhalation administration, injection administration and topical administration.

A method for treating lung injury-related diseases of a subject includes administrating the above triacetyl andrographolide nanocrystal or the above triacetyl andrographolide nanocrystal preparation to the subject, wherein oral administration is conducted 100-200 mg/time, 2-3 times/day, and inhalation administration is conducted 10-20 mg/time, 2-3 times/day.

The lung injury-related diseases include chronic obstructive pulmonary disease. The triacetyl andrographolide nanocrystal or the preparation thereof can significantly improve the pulmonary ventilation function, inhibit pathological changes in pulmonary alveoli and airway walls, and down-regulate lung tissue protease levels. It can significantly reduce the number of inflammatory cells and secretion of inflammatory factors in the lungs, and inhibit the infiltration of inflammatory cells in lung tissue.

A method for preparing the above triacetyl andrographolide nanocrystal includes the following steps:
(1) dissolving a drug triacetyl andrographolide in an organic solvent to obtain a drug solution, and dissolving a stabilizer in water to obtain a stabilizer solution;
(2) adding the drug solution in the stabilizer solution and dispersing the mixture by high-speed shear dispersion to obtain a coarse drug suspension;
(3) subjecting the coarse drug suspension to high-pressure homogenization to obtain a nanocrystal suspension of the triacetyl andrographolide; and
(4) adding an excipient to the nanocrystal suspension, and preparing the triacetyl andrographolide nanocrystal by spray-drying.

The organic solvent in step (1) is selected from one of ethanol and acetone, or a mixture thereof, and the stabilizer is selected from one or more of soybean phospholipids, Poloxamer 188, hydroxypropyl methylcellulose E5, sodium dodecyl sulfate, polyvinylpyrrolidone K30 and Tween 80; and the excipient in step (4) is selected from one or a mixture of lactose, mannitol and glucose.

The stabilizer in step (1) is preferably one or a mixture of the soybean phospholipids and the Poloxamer 188; and the excipient in step (4) is preferably one or a mixture of the lactose and the mannitol.

In step (1), a mass volume ratio of the drug triacetyl andrographolide to the organic solvent is (0.20 to 0.40 g): 1 mL;
in step (2), a volume ratio of the drug solution to the stabilizer solution is 1:20 to 1:50, a mass ratio of the triacetyl andrographolide to the stabilizer in the coarse drug suspension is 1:1 to 5:1, and high-speed shear conditions are a shear stirring speed of 5000 to 10000 rpm and a shear time of 5 to 10 min;
in step (3), during high-pressure homogenization, a homogenizing pressure is 10000 to 20000 psi, and a number of homogenization is 10 to 20 times; and
in step (4), a mass volume ratio of the excipient to the nanocrystal suspension of the triacetyl andrographolide is (0.03 to 0.10 g): 1 mL.

Process parameters during spray-drying in step (4) are: an inlet temperature of 60° C. to 70° C., an outlet temperature of 30° C. to 40° C., a spray pressure of 0.1 to 0.2 Mpa, a sample injection rate of 1.0 to 2.0 mL/min, and a drying air volume of 0.5 to 1.0 m³/min.

The present disclosure has the following beneficial effects.
(1) The triacetyl andrographolide nanocrystal suspension is prepared by adopting a high-speed shear anti-solvent method in combination with a high-pressure homogenization method, the drug nanocrystal is prepared further through spray-drying, and the conventional excipients in the art may be added into the obtained nanocrystal to prepare multiple dosage forms such as the solid preparation, the liquid preparation and the semi-solid preparation, thereby meeting drug use requirements of different administration routes. The nanocrystal has the advantages of high drug loading, low use amount of the organic solvent, controllable environment pollution, mature and stable preparation process, easy production scaling-up and the like.
(2) Compared to active pharmaceutical ingredients, the triacetyl andrographolide nanocrystal may improve dissolution performance of drugs and remarkably increase the bioavailability of the drugs.
(3) Compared to the active pharmaceutical ingredients, the triacetyl andrographolide nanocrystal has significant pharmaceutical effects for treating the lung injury-related diseases including the chronic obstructive pulmonary disease, the drug use dosage is reduced, preparation processing is facilitated, and drug use compliance and druggability are good.

### BRIEF DESCRIPTION OF THE DRAWINGS

To illustrate the technical solutions in examples of the present disclosure or in the related art more clearly, the accompanying drawings required for describing the examples or the related art are briefly introduced below. Apparently, the accompanying drawings in the following description only show some examples of the present disclosure, and those of ordinary skill in the art may still derive other accompanying drawings from these accompanying drawings without creative efforts.

FIG. 1 is appearance of a suspension of a triacetyl andrographolide nanocrystal prepared according to Example 1 and a transmission electron microscopy image of the nanocrystal.

FIG. 2 is an X-ray diffraction diagram of powder of a triacetyl andrographolide nanocrystal prepared according to Example 1.

FIG. 3 is a differential scanning calorimetry (DSC) graph of a triacetyl andrographolide nanocrystal prepared according to Example 1 vs an active pharmaceutical ingredient.

FIG. 4 is an infrared (IR) spectrum of a triacetyl andrographolide nanocrystal prepared according to Example 1 vs an active pharmaceutical ingredient.

FIG. 5 is an in-vitro release graph of a triacetyl andrographolide nanocrystal prepared according to Example 1 vs an active pharmaceutical ingredient in a simulated pulmonary fluid.

FIG. 6 is a plasma concentration-time graph in rat intragastric administration of a triacetyl andrographolide nanocrystal prepared according to Example 1 vs an active pharmaceutical ingredient.

FIG. 7 is a diagram of effects of intragastric administration of a triacetyl andrographolide nanocrystal on lung injury of acute lung injury mice constructed by LPS intratracheal instillation, wherein, in the diagram, *** is p<0.001, compared with a control group; #is p<0.05, and ##is p<0.01, compared with a model group; and & is p<0.05, compared with an active pharmaceutical ingredient group.

FIG. 8 is a diagram of effects of intragastric administration of a triacetyl andrographolide nanocrystal on inflammatory cells and inflammatory factors in a BALF of acute lung injury mice constructed by LPS intratracheal instillation, wherein, in the diagram, *** is p<0.001, compared with a control group; ###is p<0.001, compared with a model group; and & is p<0.05, && is p<0.01 and &&& is p<0.001, compared with an active pharmaceutical ingredient group.

FIG. 9 is a diagram of effects of intragastric administration of a triacetyl andrographolide nanocrystal on lung injury of acute lung injury mice constructed by poly(I:C) intratracheal instillation, wherein, in the diagram, *** is p<0.001, compared with a control group; #is p<0.05, and ##is p<0.01, compared with a model group; and & is p<0.05, compared with an active pharmaceutical ingredient group.

FIG. 10 is a diagram of effects of intragastric administration of a triacetyl andrographolide nanocrystal on inflammatory cells and inflammatory factors in a BALF of acute lung injury mice constructed by poly(I:C) intratracheal instillation, wherein, in the diagram, ** is p<0.001, compared with a control group; ###is p<0.001, compared with a model group; and & is p<0.05, && is p<0.01, and &&& is p<0.001, compared with an active pharmaceutical ingredient group.

FIG. 11 is a diagram of effects of a triacetyl andrographolide nanocrystal on supernatant inflammatory factors of RAW264.7 cells of mice stimulated by a combination of cigarette smoke extract and LPS, wherein, in the diagram, ** is p<0.01 and *** is p<0.001, compared with a control group; #is p<0.05, ##is p<0.01 and ###is p<0.001, compared with a model group; and & is p<0.05, && is p<0.01 and &&& is p<0.001, compared with an active pharmaceutical ingredient group.

FIG. 12 is a diagram of effects of intragastric administration of a triacetyl andrographolide nanocrystal on lung functions of chronic obstructive pulmonary disease mice constructed by a combination of cigarette smoke and LPS, wherein, in the diagram, is p<0.001, compared with a control group; ###is p<0.001, compared with a model group; and & is p<0.05 and &&& is p<0.001, compared with a dexamethasone group, and $ is p<0.05 and $$$ is p<0.001, compared with an active pharmaceutical ingredient group.

FIG. 13 is a diagram of effects of intragastric administration of a triacetyl andrographolide nanocrystal on lung injury of chronic obstructive pulmonary disease mice constructed by a combination of cigarette smoke and LPS, wherein, in the diagram, ** is p<0.01 and *** is p<0.001, compared with a control group; #is p<0.05, ##is p<0.01 and ###is p<0.001, compared with a model group; and & is p<0.05 and &&& is p<0.001, compared with an active pharmaceutical ingredient group.

FIG. 14 is a diagram of effects of intragastric administration of a triacetyl andrographolide nanocrystal on inflammatory cells and inflammatory factors in a BALF and protease in lung tissue of chronic obstructive pulmonary disease mice constructed by a combination of cigarette smoke and LPS, wherein, in the diagram, ** is p<0.01 and *** is p<0.001, compared with a control group; #is p<0.05, ##is p<0.01 and ###is p<0.001, compared with a model group; and & is p<0.05, && is p<0.01 and &&& is p<0.001, compared with an active pharmaceutical ingredient group.

FIG. 15 is a diagram of effects of suspended nebulization pulmonary inhalation administration of a triacetyl andrographolide nanocrystal on lung functions of chronic obstructive pulmonary disease mice constructed by a combination of cigarette smoke and LPS, wherein, in the diagram, * is p<0.05, ** is p<0.01 and *** is p<0.001, compared with a control group; #is p<0.05, ##is p<0.01 and ###is p<0.001, compared with a model group; and & is p<0.05, && is p<0.01 and &&& is p<0.001, compared with a budesonide group.

FIG. 16 is a diagram of effects of suspended nebulization pulmonary inhalation administration of a triacetyl andrographolide nanocrystal on lung injury of chronic obstructive pulmonary disease mice constructed by a combination of cigarette smoke and LPS, wherein, in the diagram, *** is p<0.001, compared with a control group; ##is p<0.01 and ###is p<0.001, compared with a model group; and && is p<0.01 and &&& is p<0.001, compared with a budesonide group.

FIG. 17 is a diagram of effects of suspended nebulization pulmonary inhalation administration of a triacetyl andrographolide nanocrystal on inflammatory cells and inflammatory factors in a BALF and protease in lung tissue of chronic obstructive pulmonary disease mice constructed by a combination of cigarette smoke and LPS, wherein, in the diagram, *** is p<0.001, compared with a control group; #is p<0.05, ##is p<0.01 and ###is p<0.001, compared with a model group; and & is p<0.05, && is p<0.01 and &&& is p<0.001, compared with a budesonide group.

### DETAILED DESCRIPTION

The technical solution in examples of the present disclosure will be clearly and completely described below in combination with the accompanying drawings in the examples of the present disclosure. Apparently, the described examples are only part of the examples of the present disclosure, not all of them. Based on the examples of the present disclosure, all other examples obtained by those of ordinary skill in the art without any creative work fall within the scope of protection of the present disclosure.

### Example 1

The present example provides a method for preparing a triacetyl andrographolide nanocrystal, including the following steps:
150 mg of a triacetyl andrographolide active pharmaceutical ingredient was weighed and placed in 0.5 mL of ethanol to be dissolved so as to obtain a drug solution, and 75 mg of soybean phospholipids were weighed and dissolved in 15 mL of purified water to be used as a stabilizer solution;
the drug solution was added in the stabilizer solution, and the mixture was dispersed by 5000 rpm high-speed shear dispersion for 5 min to prepare a coarse suspension;
the coarse suspension was placed in a high-pressure homogenizer and pre-homogenized by being circulated 5 times under pressures of 3000, 5000 and 7000 psi respectively, and then high-pressure homogenization was performed by setting a high-pressure homogenizing pressure to be 15000 psi to conduct homogenization 15 times, so as to obtain a nanocrystal suspension; and
lactose was added to the above nanocrystal suspension as an excipient, with a use amount being a mass volume ratio of 0.1 g:1 mL, and spray-drying was carried out according to following process parameters: an inlet temperature of 65° C., an outlet temperature of 35° C., a spray pressure of 0.1 Mpa, a sample injection rate of 1.0 mL/min, and a drying air volume of 0.75 m³/min, so as to prepare the triacetyl andrographolide nanocrystal, wherein, a particle size of drug particles in a suspension obtained by adding water to the nanocrystal for redissolution, was 461.6 nm, and a PDI was 0.094.

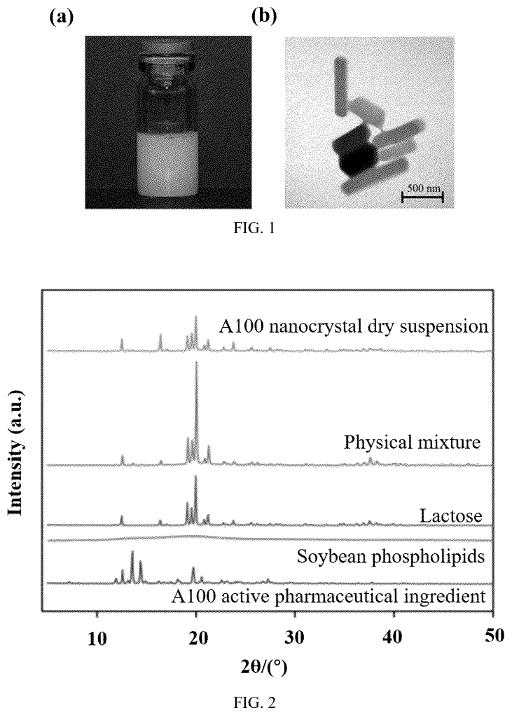

Water was added to the obtained nanocrystal for redissolution to form a suspension, the suspension was placed in a transparent vial, an appearance photo was taken (see FIG. 1a), and the nanocrystal suspension is milk white in appearance, uniform in dispersion and stable. 10 μL of the above suspension was sucked and dropwise added onto a clean copper screen covered with a carbon film, surplus samples were sucked dry using filter paper, the copper screen was placed at a room temperature to be dried, and finally the dried copper screen was placed under a transmission electron microscope to observe the form of nanocrystal particles and take a photo (see FIG. 1b). It can be seen through the transmission electron microscope that, the nanocrystal is in a shape of a short rod, is not gathered, has a particle size of about 450 nm and is distributed uniformly.

The triacetyl andrographolide nanocrystal (no lactose was added during spray-drying), a triacetyl andrographolide active pharmaceutical ingredient, soybean phospholipids and a physical mixture of triacetyl andrographolide and soybean phospholipids were taken respectively, and uniformly filled grooves of a glass sample plate, and the glass sample plate was placed on a sample table of a diffraction instrument to perform scanning. Testing conditions: a radioactive source was Cu-ka, a tube current was 40 mA, a tube voltage was 40 kV, a scanning speed was 2.5°/min, a step length was 0.03°, a 2θ angle scanning rage was 5° to 60°, an XRD diffraction diagram was recorded, and crystal form changes were judged according to changes of positions and intensities of diffraction peaks of various groups of samples, see FIG. 2 for the results.

It is shown in an XRD spectrum in FIG. 2 that, the soybean phospholipids have a diffusion peak, without characteristic diffraction peaks; and the triacetyl andrographolide nanocrystal and the triacetyl andrographolide active pharmaceutical ingredient both exhibit obvious crystalline diffraction peaks at 2θ values of 11.71°, 11.83°, 12.38°, 12.50°, 13.46°, 14.37°, 19.63° and 20.48°, indicating that the nanocrystal drug and the active pharmaceutical ingredient have the same crystal form features.

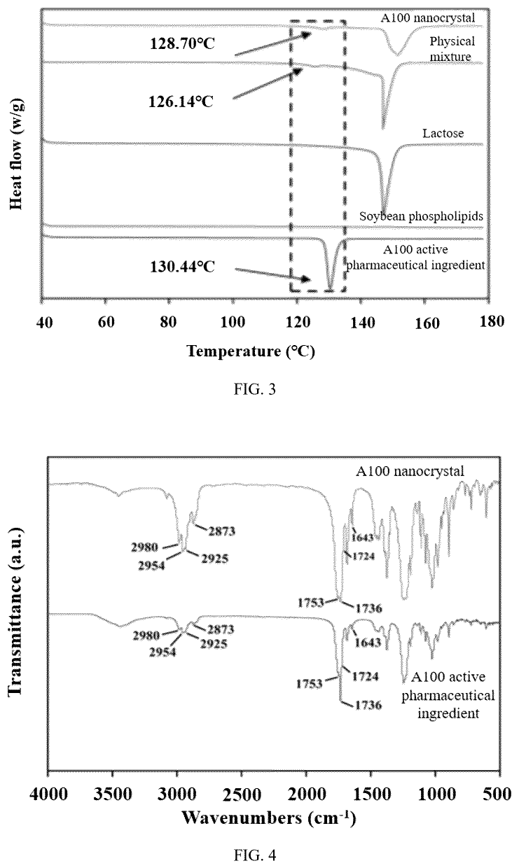

It is shown in a DSC graph in FIG. 3 that, the triacetyl andrographolide active pharmaceutical ingredient has an endothermic peak at 130.44° C.; a DSC curve of the soybean phospholipids has no endothermic peak within this range, indicating that the soybean phospholipids have no crystal structure and are in an amorphous state; and the triacetyl andrographolide nanocrystal and a physical mixture have endothermic peaks at 128.70° C. and 126.14° C. respectively in addition to containing an absorption peak of lactose, indicating that drug particles in the triacetyl andrographolide nanocrystal still exist in a crystal form.

It is shown in an IR graph in FIG. 4 that, the triacetyl andrographolide active pharmaceutical ingredient and the triacetyl andrographolide nanocrystal both have absorption peaks at 2980, 2954, 2925, and 2873 cm⁻¹, showing the presence of a methyl group; have absorption peaks at 1753 cm⁻¹, showing the presence of an unsaturated lactone carbonyl group in a ring; have absorption peaks at 1736 cm⁻¹, showing the presence of an ester group; have absorption peaks at 1724 cm⁻¹, showing the presence of a five-membered lactone ring; and have absorption peaks at 1643 cm⁻¹, showing the presence of double bonds. The position of a main characteristic peak of the triacetyl andrographolide nanocrystal is similar to that of the active pharmaceutical ingredient. The results indicate that the chemical structure does not vary after the triacetyl andrographolide nanocrystal is prepared.

### Example 2

The present example provides a method for preparing a triacetyl andrographolide nanocrystal, including the following steps:
400 mg of a triacetyl andrographolide active pharmaceutical ingredient was weighed and placed in 1.0 mL of ethanol to be dissolved so as to obtain a drug solution, and 100 mg of Poloxamer 188 was weighed and dissolved in 20 mL of purified water to be used as a stabilizer solution;
the drug solution was added in the stabilizer solution, and the mixture was dispersed by 10000 rpm high-speed shear dispersion for 5 min to prepare a coarse suspension;
the coarse suspension was placed in a high-pressure homogenizer and pre-homogenized by being circulated 5 times under pressures of 3000, 5000 and 7000 psi respectively, and then high-pressure homogenization was performed by setting a high-pressure homogenizing pressure to be 20000 psi to conduct homogenization 20 times, so as to obtain a nanocrystal suspension; and
mannitol was added to the above nanocrystal suspension as an excipient, with a use amount being a mass volume ratio of 0.05 g:1 mL, and spray-drying was carried out according to following process parameters: an inlet temperature of 65° C., an outlet temperature of 35° C., a spray pressure of 0.1 Mpa, a sample injection rate of 1.5 mL/min, and a drying air volume of 1.0 m³/min, so as to prepare the triacetyl andrographolide nanocrystal. A particle size of drug particles in a suspension obtained by redissolving the nanocrystal in water was 436.3 nm, and a PDI was 0.128.

### Example 3

The present example provides a method for preparing a triacetyl andrographolide nanocrystal, including the following steps:
200 mg of a triacetyl andrographolide active pharmaceutical ingredient was weighed and placed in 0.5 mL of acetone to be dissolved so as to obtain a drug solution, and 160 mg of soybean phospholipids and 40 mg of Poloxamer 188 were weighed and dissolved in 20 mL of purified water to be used as a stabilizer solution;
the drug solution was added in the stabilizer solution, and the mixture was dispersed by 7500 rpm high-speed shear dispersion for 10 min to prepare a coarse suspension;
the coarse suspension was placed in a high-pressure homogenizer and pre-homogenized by being circulated 5 times under pressures of 3000, 5000 and 7000 psi respectively, and then high-pressure homogenization was performed by setting a high-pressure homogenizing pressure to be 15000 psi to conduct homogenization 10 times, so as to obtain a nanocrystal suspension; and
lactose and mannitol (use amounts both being a mass volume ratio of 0.015 g:1 mL) were added to the above nanocrystal suspension as an excipient, and spray-drying was carried out according to following process parameters: an inlet temperature of 60° C., an outlet temperature of 40° C., a spray pressure of 0.2 Mpa, a sample injection rate of 2.0 mL/min, and a drying air volume of 1.0 m³/min, so as to prepare the triacetyl andrographolide nanocrystal. A particle size of drug particles in a suspension obtained by adding water to the nanocrystal for redissolution was 445.7 nm, and a PDI was 0.142.

### Example 4

The present example provides a method or preparing a triacetyl andrographolide nanocrystal, including the following steps:
200 mg of a triacetyl andrographolide active pharmaceutical ingredient was weighed and placed in 1 mL of a mixed solution of ethanol and acetone (a volume ratio of 8:1) to be dissolved so as to obtain a drug solution, and 32 mg of soybean phospholipids and 8 mg of Tween 80 were weighed and dissolved together in 40 mL of purified water to be used as a stabilizer solution;
the drug solution was added in the stabilizer solution, and the mixture was dispersed by 5000 rpm high-speed shear dispersion for 10 min to prepare a coarse suspension;
the coarse suspension was placed in a high-pressure homogenizer and pre-homogenized by being circulated 5 times under pressures of 3000, 5000 and 7000 psi respectively, and then high-pressure homogenization was performed by setting a high-pressure homogenizing pressure to be 10000 psi to conduct homogenization 20 times, so as to obtain a nanocrystal suspension; and
a mixture of lactose and mannitol (use amounts being mass volume ratios of 0.04 g:1 mL and 0.01 g:1 mL respectively) was added to the above nanocrystal suspension as an excipient, and spray-drying was carried out according to following process parameters: an inlet temperature of 70° C., an outlet temperature of 30° C., a spray pressure of 0.2 Mpa, a sample injection rate of 2.0 mL/min, and a drying air volume of 1.0 m³/min, so as to prepare the triacetyl andrographolide nanocrystal. A particle size of drug particles in a suspension obtained by adding water to the nanocrystal for redissolution was 485.1 nm, and a PDI was 0.135.

### Example 5

The present example provides a method for preparing a triacetyl andrographolide nanocrystal, including the following steps:
300 mg of a triacetyl andrographolide active pharmaceutical ingredient was weighed and placed in 0.75 mL of a mixed solution of ethanol and acetone (a volume ratio of 1:1) to be dissolved so as to obtain a drug solution, and 100 mg of soybean phospholipids were weighed and dissolved in 22.5 mL of purified water to be used as a stabilizer solution;
the drug solution was added in the stabilizer solution, and the mixture was dispersed by 5000 rpm high-speed shear dispersion for 10 min to prepare a coarse suspension;
the coarse suspension was placed in a high-pressure homogenizer and pre-homogenized by being circulated 5 times under pressures of 3000, 5000 and 7000 psi respectively, and then high-pressure homogenization was performed by setting a high-pressure homogenizing pressure to be 20000 psi to conduct homogenization 15 times, so as to obtain a nanocrystal suspension; and
lactose and mannitol (use amounts being mass volume ratios of 0.04 g:1 mL and 0.01 g:1 mL respectively) were weighed and added to the above nanocrystal suspension as an excipient, and spray-drying was carried out according to following process parameters: an inlet temperature of 70° C., an outlet temperature of 30° C., a spray pressure of 0.2 Mpa, a sample injection rate of 1.0 mL/min, and a drying air volume of 0.5 m³/min, so as to prepare the triacetyl andrographolide nanocrystal. A particle size of drug particles in a suspension obtained by adding water to the nanocrystal for redissolution was 460.9 nm, and a PDI was 0.130.

### Example 6

The present example provides a method for preparing a triacetyl andrographolide nanocrystal, including the following steps:
240 mg of a triacetyl andrographolide active pharmaceutical ingredient was weighed and placed in 1.0 mL of ethanol to be dissolved so as to obtain a drug solution, and 120 mg of a stabilizer (soybean phospholipids and Poloxamer 188 with a mass ratio of 2:1) was weighed and dissolved in 40 mL of purified water to be used as a stabilizer solution;
the drug solution was added in the stabilizer solution, and the mixture was dispersed by 7000 rpm high-speed shear dispersion for 8 min to prepare a coarse suspension;
the coarse suspension was placed in a high-pressure homogenizer and pre-homogenized by being circulated 5 times under pressures of 3000, 5000 and 7000 psi respectively, and then high-pressure homogenization was performed by setting a high-pressure homogenizing pressure to be 20000 psi to conduct homogenization 15 times, so as to obtain a nanocrystal suspension; and
lactose (a mass volume ratio of 0.03 g:1 mL) was added to the above nanocrystal suspension as an excipient, and spray-drying was carried out according to following process parameters: an inlet temperature of 65° C., an outlet temperature of 35° C., a spray pressure of 0.1 Mpa, a sample injection rate of 1.0 mL/min, and a drying air volume of 0.75 m³/min, so as to prepare the triacetyl andrographolide nanocrystal. A particle size of drug particles in a suspension obtained by adding water to the nanocrystal for redissolution was 417.5 nm, and a PDI was 0.118.

The triacetyl andrographolide nanocrystal prepared in Examples 2-6 was characterized to obtain the appearance of a suspension and the transmission electron microscopy image of the nanocrystal, the X-ray diffraction diagram of the powder of the triacetyl andrographolide nanocrystal, the DSC graph and the IR graph, which were similar to corresponding characterization results in Example 1.

### Application Example 1

### Preparation of Triacetyl Andrographolide Nanocrystal Dry Suspension

2.0 g of the triacetyl andrographolide nanocrystal obtained in Example 1 was taken, and 0.05 g of sodium carboxymethyl cellulose was added and mixed fully and uniformly to obtain a triacetyl andrographolide nanocrystal dry suspension. The nanocrystal dry suspension could be dispersed uniformly after water was added, and no obvious settlement layer was formed after 30 min of standing.

### Application Example 2

### Preparation of Triacetyl Andrographolide Nanocrystal Dispersible Tablets

2.0 g of the triacetyl andrographolide nanocrystal obtained in Example 1 was taken, 0.14 g of sodium carboxymethyl starch and 0.06 g of colloidal silicon dioxide were added and mixed fully and uniformly, and tabletting was performed by adopting a 9 mm shallow-arc punching die, wherein a tablet weight was 250 mg. Nanocrystal dispersible tablets were bright and clean in tablet surface and complete, and the dispersible tablets could all collapse within 1 min and pass through a screen in a dispersion uniformity test.

### Experimental Example 1: Determination of In-Vitro Release Degree of Triacetyl Andrographolide Nanocrystal in Simulated Lungs

With the triacetyl andrographolide nanocrystal prepared in Example 1 as an experimental subject, an activated dialysis bag with a molecular weight cut-off of 8000-14000 Da was taken, 5 mL of a simulated pulmonary fluid was added into the dialysis bag, the triacetyl andrographolide nanocrystal (0.5 mg by triacetyl andrographolide) was precisely weighed and dispersively suspended in the above simulated pulmonary fluid, two ends were tightened, then the dialysis bag was put into a conical flask containing 100 mL of a simulated pulmonary fluid in-vitro release medium (composition of the medium as shown in Table 1), the conical flask was placed in a constant-temperature gas bath shaker (37° C., 120 r/min), 2 mL of a released liquor was taken at each of different time points (0.5 h, 1 h, 2 h, 4 h, 6 h, 8 h, 12 h, 24 h, 36 h and 48 h) and was filtered through a 0.22 μm filter membrane, subsequence filtrates were taken to determine drug concentrations by adopting an HPLC method, and at the same time, the release medium was supplemented with 2 mL of an isothermal simulated pulmonary fluid. A cumulative release percentage was calculated according to determined results, and a release curve of the nanocrystal was drawn. Meanwhile, a triacetyl andrographolide active pharmaceutical ingredient with the same amount as the above nanocrystal used for release degree determination was taken, the same operation as the method for determining the release degree of the nanocrystal was performed, and a release curve of the active pharmaceutical ingredient was drawn.

An experiment was conducted on the active pharmaceutical ingredient with the same effective content according to the above method.

TABLE 1
Composition of in-vitro release medium
of simulated pulmonary fluid

| Composition | Concentration (g/L) | pH |
|---|---|---|
| Magnesium chloride | 0.2033 | 7.4 |
| (MgCl₂•6H₂O) | | |
| Sodium chloride (NaCl) | 6.0913 | |
| Potassium chloride (KCl) | 0.2982 | |
| Sodium hydrogen | 0.2501 | |
| phosphate | | |
| (Na₂HPO₄•12H₂O) | | |
| Sodium sulfate (Na₂SO₄) | 0.0710 | |
| Calcium chloride (CaCl₂) | 0.2775 | |
| Sodium acetate | 0.5744 | |
| (NaC₂H₃O₂•3H₂O) | | |
| Sodium bicarbonate | 2.6043 | |
| (NaHCO₃) | | |
| Sodium citrate | 0.0970 | |
| (C₆H₅Na₃O₇) | | |

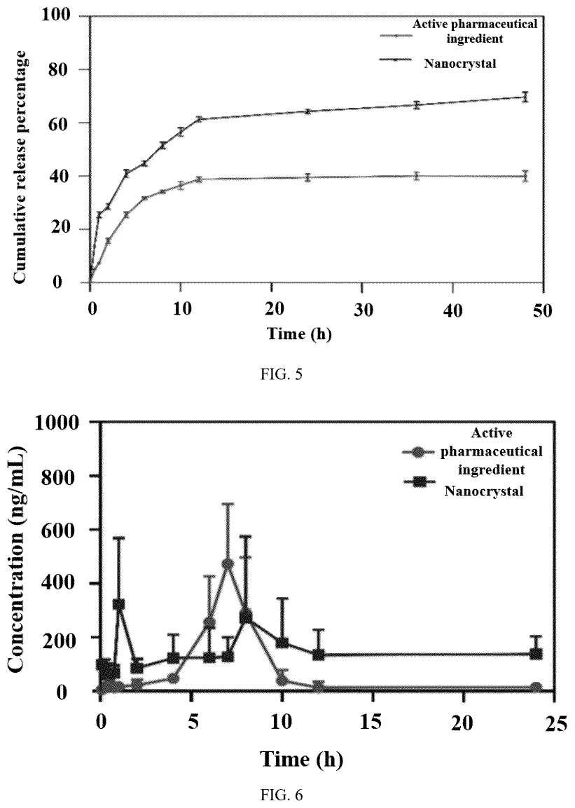

The results are shown in FIG. 5, compared to the active pharmaceutical ingredient, an in-vitro drug release speed of the triacetyl andrographolide nanocrystal is obviously higher, the cumulative release percentage at 12 h is increased from 39% to 64%, and the cumulative release percentage of the nanocrystal at 48 h is increased from 42% to 69%.

### Experimental Example 2: Determination of Bioavailability of Triacetyl Andrographolide Nanocrystal in Intragastric Administration of Rats

The triacetyl andrographolide nanocrystal obtained in Example 1 was adopted, and a total of 12 SD rats were used as test objects (half female and half male, 200±20 g).

The rats were randomly divided into 2 groups (n=6, half female and half male) by body weight before a test was started. The test was carried out after fasting without water-deprivation for 12 h, one group was fed with a triacetyl andrographolide active pharmaceutical ingredient suspension (dosage of 200 mg/kg) via intragastric administration, and the other group was fed with a triacetyl andrographolide nanocrystal suspension (50 mg/kg by triacetyl andrographolide) via intragastric administration.

Blank blood was collected respectively before administration, 200 μL of blood was collected from fundus oculi venous plexus at stipulated time (0.083 h, 0.25 h, 0.50 h, 0.75 h, 1 h, 2 h, 4 h, 6 h, 7 h, 8 h, 10 h, 12 h, 24 h respectively), the blood was placed in heparinized blood collection tubes and centrifuged at 5000 rpm for 10 min, plasma samples were taken, after treatment, drug concentrations in the plasma samples were measured, plasma concentration-time curves were drawn, and by adopting WinNonlin® 6.4 version (Pharsight Corporation, USA) software, relevant pharmacokinetic parameters were calculated according to non-compartment models, including: AUC₀₋ₜ, MRT, CL, V_{d}, Cₘₐₓ and Tₘₐₓ, see FIG. 6 and Table 2 for the results.

With an active pharmaceutical ingredient as reference, relative bioavailability of the triacetyl andrographolide nanocrystal was calculated according to the following formula:

$${F}_{rel} = \frac{{AUC}_{0 - τ , nanocrystal}}{{AUC}_{0 - τ , active pharmaceutical ingredient}} \times \frac{{Dose}_{active pharmaceutical ingredient}}{{Dose}_{nanocrystal}} \times 100 \%$$

where AUC_{0-t, nanocrystal} and AUC_{0-t, active pharmaceutical ingredient} corresponded to areas under the plasma concentration-time curves at a moment 0-t of the triacetyl andrographolide nanocrystal and the active pharmaceutical ingredient respectively; and

Dose_{active pharmaceutical ingredient} and Dose_{nanocrystal} corresponded to intragastric administration dosages of the triacetyl andrographolide active pharmaceutical ingredient and the nanocrystal respectively.

TABLE 2
Pharmacokinetic parameters of triacetyl andrographolide
nanocrystal and active pharmaceutical ingredient
in intragastric administration of rats

| | | Active pharmaceutical | Nanocrystal |
|---|---|---|---|
| Parameter | Unit | ingredient (200 mg/kg) | (50 mg/kg) |
| AUC₍₀₋ₜ₎ | h*ng/mL | 1924 ± 277.9 | 2023.72 ± 738.55 |
| MRT₍₀₋ₜ₎ | h | 8.07 ± 0.22 | 9.18 ± 2.08 |
| T_{1/2} | h | 4.79 ± 1.81 | 16.77 ± 13.79 |
| Tₘₐₓ | h | 6.67 ± 0.47 | 1.15 ± 1.61 |
| Cₘₐₓ | ng/mL | 495.1 ± 172.6 | 365.58 ± 59.72 |
| V_{d}/F | mL/kg | 0.80 ± 0.36 | 0.28 ± 0.14 |
| CL/F | mL/h/kg | 0.12 ± 0.04 | 0.02 ± 0.01 |
| Fᵣₑₗ | % | | 421 ± 154 |

where MRT₍₀₋ₜ₎ is a mean value of in-vivo retention time of drug molecules, representing time required for eliminating 63.2% of drugs in vivo;
T_{1/2} is a terminal elimination half-life, which is time required for a terminal phase plasma concentration to decrease by half;
Tₘₐₓ is time required to reach a peak concentration of drug after administration, this parameter reflects a speed at which a drug enters the body, and the faster the absorption rate, the shorter the time to peak;
Cₘₐₓ is a peak concentration of drug, the highest value of a plasma concentration occurring after administration, and this parameter is an important indicator reflecting a rate and degree of drug absorption in the body;
V_{d} is a proportionality constant between a drug dosage in the body and a plasma concentration when a drug reaches dynamic equilibrium in the body, usually with Las the unit, and this parameter reflects the degree of a distribution range of the drug in the body, with higher values indicating the wider distribution; and an apparent volume of distribution is numerically obtained by a ratio of a clearance rate to a terminal elimination rate;
F is bioavailability, a measure for a speed and degree at which a drug is absorbed into blood circulation, and is an important indicator for evaluating a degree of drug absorption; and the bioavailability may be divided into absolute bioavailability and relative bioavailability, the former is used to compare an absorption difference between two administration routes, while the latter is used to evaluate an absorption difference between two preparations; and
CL is a clearance rate, a value of an apparent volume of distribution of drugs cleared from the body per unit time, usually with L/h as the unit, and this parameter is an important parameter reflecting the body's drug disposal characteristics and is closely related to physiological factors; and the clearance rate is obtained according to a ratio of a dosage to AUC_{(0-∞)}.

It can be seen from FIG. 6 that, the nanocrystal can significantly increase the oral bioavailability of rat intragastric administration of the triacetyl andrographolide, and compared to the active pharmaceutical ingredient, oral relative bioavailability of the triacetyl andrographolide nanocrystal is 421%.

### Experimental Example 3: Efficacy Evaluation of Intragastric Administration of Triacetyl Andrographolide Nanocrystal in Treatment of Acute Lung Injury Mouse Models Constructed by LPS Intratracheal Instillation

A triacetyl andrographolide nanocrystal dry suspension was prepared according to Application Example 1, and male KM mice were selected for experiments, 70 mice in total (20±2 g).

Before the start of testing, the mice were randomly divided into 7 groups (n=10) by body weight, including a control group, a model group, a dexamethasone group (1 mg/kg), an active pharmaceutical ingredient group (200 mg/kg), and a nanocrystal group (50, 100 and 200 mg/kg by triacetyl andrographolide).

On day 1, an LPS solution (5 mg/kg) was intratracheally instilled to the mice in the model group and the administration group, and normal saline of a corresponding volume was intratracheally instilled to the mice in the control group. Intragastric administration was conducted on the administration group from day 2 to day 6 once a day, and 0.5% CMC-Na (0.1 mL/g) of a corresponding volume was given to the mice in the control group and the model group intragastrically. The experiment was ended on day 7, alveolar lavage fluids of the mice were collected and centrifuged in a low-temperature centrifuge at 3000 rpm for 10 min, supernatants and cell pellets were taken, and an inflammatory factor level and the number of inflammatory cells were detected by using an ELISA method and a Giemsa staining method respectively. Whole lungs of the mice were taken additionally and weighed, and a viscera index was calculated. A left lung wet weight was obtained by weighing the left lung, the left lung was placed into a 1.5 mL centrifuge tube which was dried to a constant weight in advance, the centrifuge tube was placed in a 60° C. oven, forced air drying was carried out for 72 h, a total weight of the centrifuge tube and a dry weight of the lung was obtained by weighing, and a wet/dry weight ratio of the left lung of each mouse was calculated. The inferior lobe of the right lung of each mouse was taken and soaked in 4% paraformaldehyde to be fixed for 24 h, paraffin sections of lung tissue of the mice were manufactured using conventional methods for dehydration, paraffin embedding, sectioning (5 μm), HE staining and mounting, and pathologic changes of the lung tissue of the mice were observed and photographed.

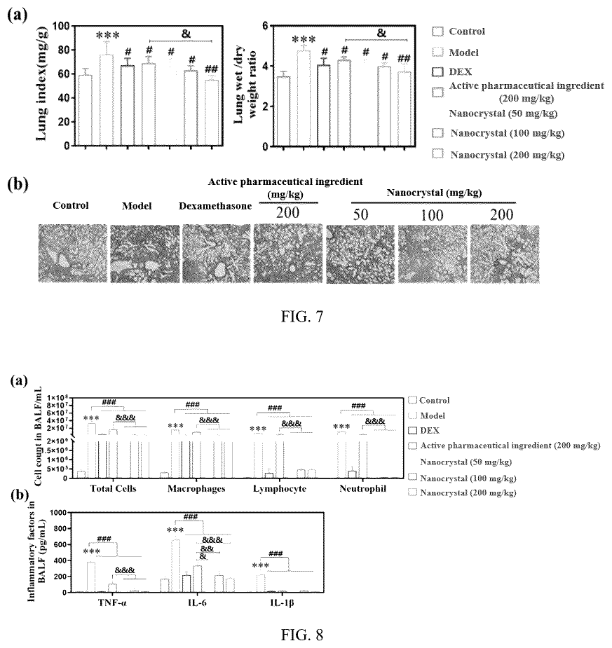

Results show that, the nanocrystals (50, 100, 200 mg/kg) significantly reduce the lung index and the lung wet/dry weight ratio of the acute lung injury mice (see FIG. 7a for the results), and obviously alleviate pulmonary injury symptoms such as alveolar wall edema, alveolar septum thickening, and inflammatory cell infiltration caused by lipopolysaccharide in the mice (see FIG. 7b for the results). The protective effect of low-dosage nanocrystals on mouse lung injury is equivalent to that of the active pharmaceutical ingredient (200 mg/kg), and the effect of nanocrystals of the same dosage is better than that of the active pharmaceutical ingredient. The nanocrystals (50, 100, 200 mg/kg) significantly inhibit the systemic inflammatory response induced by LPS in the mice, manifested by a significant decrease in total cell count, lymphocyte count, neutrophil count and levels of TNF-α, IL-6, and IL-1β in the mouse alveolar lavage fluid. Except for IL-1B, the effects of various dosages of nanocrystals on the above indicators are significantly better than those of the active pharmaceutical ingredient (see FIG. 8 for the results), and the effects of the nanocrystals are equivalent to those of dexamethasone.

### Experimental Example 4: Efficacy Evaluation of Intragastric Administration of Triacetyl Andrographolide Nanocrystal in Treatment of Acute Lung Injury Mouse Models Constructed by Poly(I:C) Intratracheal Instillation

A triacetyl andrographolide nanocrystal dry suspension was prepared according to Application Example 1, and male C57BL/6 mice were selected for experiments, 70 mice in total (20±2 g).

Before the start of testing, the mice were randomly divided into 7 groups (n=10) by body weight, including a control group, a model group, a prednisone acetate group (3 mg/kg), an active pharmaceutical ingredient group (200 mg/kg), and a nanocrystal group (50, 100 and 200 mg/kg by triacetyl andrographolide).

On day 1, a poly(I:C) solution (20 mg/kg) was intratracheally instilled to the model group and the administration group, and normal saline of a corresponding volume was intratracheally instilled to the mice in the control group.

Intragastric administration was conducted on the administration group from day 2 to day 6 once a day, and 0.5% CMC-Na (0.1 mL/g) of a corresponding volume was given to the mice in the control group and the model group intragastrically.

The experiment was ended on day 7, alveolar lavage fluids of the mice were collected and centrifuged in a low-temperature centrifuge at 3000 rpm for 10 min, supernatants and cell pellets were taken, and an inflammatory factor level and the number of inflammatory cells were detected by using an ELISA method and a Giemsa staining method respectively.

Whole lungs of the mice were taken additionally and weighed, and a viscera index was calculated. A left lung wet weight was obtained by weighing the left lung, the left lung was placed into a 1.5 mL centrifuge tube which was dried to a constant weight in advance, the centrifuge tube was placed in a 60° C. oven, forced air drying was carried out for 72 h, a total weight of the centrifuge tube and a dry weight of the lung was obtained by weighing, and a wet/dry weight ratio of the left lung of each mouse was calculated. The inferior lobe of the right lung of each mouse was taken and soaked in 4% paraformaldehyde to be fixed for 48 h, paraffin sections of lung tissue of the mice were manufactured using conventional methods for dehydration, paraffin embedding, sectioning (5 μm), HE staining and mounting, and pathologic changes of the lung tissue of the mice were observed and photographed.

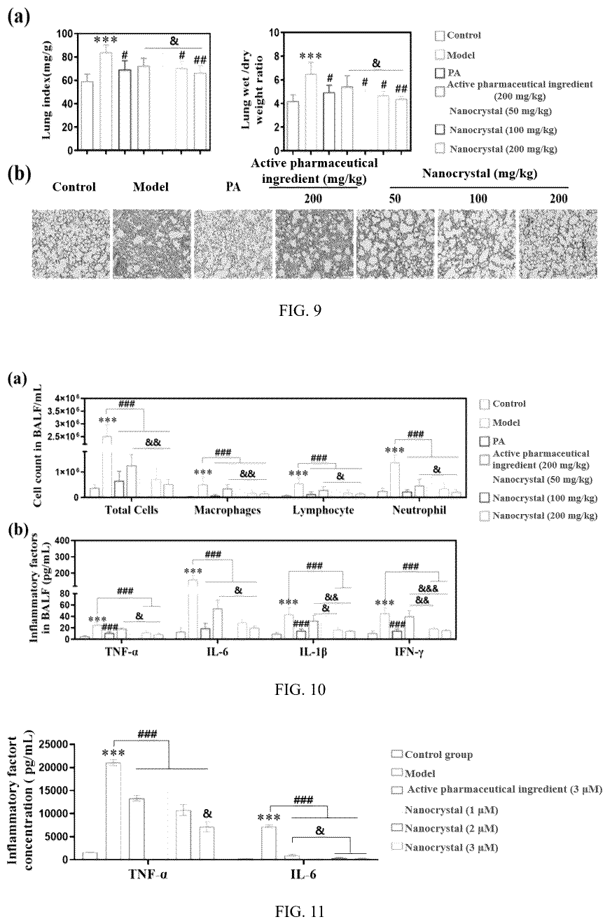

Results show that, the triacetyl andrographolide nanocrystals (50, 100, 200 mg/kg) significantly reduce the lung index and the lung wet/dry weight ratio of the acute lung injury mice (see FIG. 9a for the results), and obviously alleviate pulmonary injury symptoms such as alveolar wall edema, alveolar septum thickening, and inflammatory cell infiltration caused by poly(I:C) in the mice (see FIG. 9b for the results).

The protective effect of low-dosage nanocrystals on mouse lung injury is equivalent to that of the active pharmaceutical ingredient (200 mg/kg), and the effect of triacetyl andrographolide nanocrystals of the same dosage is better than that of the active pharmaceutical ingredient.

The triacetyl andrographolide nanocrystals (50, 100, 200 mg/kg) significantly inhibit the pulmonary inflammatory response in the mice caused by poly(I:C), manifested by a significant decrease in total cell count, lymphocyte count, neutrophil count, macrophage count and levels of TNF-α, IL-6, IL-1β and IFN-γ in the alveolar lavage fluids of the mice, which is in positive correlation with the dosage (see FIG. 10 for the results).

### Experimental Example 5: Efficacy Evaluation of Triacetyl Andrographolide Nanocrystal in Inhibition of Secretion of Inflammatory Factors by Macrophage RAW264.7 of Mice Stimulated by Cigarette Smoke Extract in Combination with LPS

Logarithmic phase RAW264.7 cells were taken and inoculated in a 6-well plate to be cultured overnight. The cells were divided into a control group, a model group, an active pharmaceutical ingredient group (3 μM), and a nanocrystal group (1, 2, 3 μM by triacetyl andrographolide), and placed in an incubator to be cultured for 1 h after corresponding drugs or solvents were added. Supernatants were discarded, and culture media containing cigarette smoke extract with a final concentration of 0.0625%, 3 μg/mL LPS and corresponding dosages of drugs or solvents were added, and placed in the incubator to continue culture for 24 h. The culture media in the 6-well plate were centrifuged at 12000 g for 5 min, supernatants were taken, and inflammatory factor levels were detected by using an ELISA method.

Results show that, the triacetyl andrographolide nanocrystals (1, 2, 3 μM) can all significantly inhibit high secretion of TNF-α and IL-6 of the RAW264.7 cells caused by the cigarette smoke extract in combination with the LPS; and the effect of low-dosage nanocrystals is equivalent to that of the active pharmaceutical ingredient (3 μM), and the effect of nanocrystals of the same dosage is better than that of the active pharmaceutical ingredient (FIG. 11).

### Experimental Example 6: Efficacy Evaluation of Intragastric Administration of Triacetyl Andrographolide Nanocrystal in Treatment of Chronic Obstructive Pulmonary Disease Mouse Models Constructed by Combination of Cigarette Smoking and LPS Intratracheal Instillation

The triacetyl andrographolide nanocrystal dry suspension obtained in Application Example 1 was used as a drug, and male KM mice were selected for experiments, 105 mice in total (20±2 g). Before the start of testing, the mice were randomly divided into 7 groups (n=15) by body weight, including a control group, a model group, a dexamethasone group (1 mg/kg), an active pharmaceutical ingredient group (200 mg/kg), and a nanocrystal group (50, 100 and 200 mg/kg by triacetyl andrographolide).

From day 1 to day 90, the animals were placed in an animal gas poisoning instrument to be smoked, with a total particle concentration of cigarette smoke kept to 500 mg/m³, the animals were smoked once a day, 1 h per time, and the animals were smoked 6 days a week.

50 μL of an LPS solution (20 μg/mL) was sprayed into the lungs of the model animals on day 91 respectively.

Intragastric administration was conducted on the administration group from day 92 to day 96 once a day, and 0.5% CMC-Na (0.1 mL/g) of a corresponding volume was given to the mice in the control group and the model group intragastrically.

The experiment was ended on day 97, tracheal intubation was performed, trachea cannulas were connected into mouse plethysmography chambers, and lung function indicators such as FEV_{0.1}, FVC, FEV_{0.1}/FVC, TLC, FRC, RI and Cydn were detected. Afterwards, chests were opened, the left lungs were irrigated by ligating hili of right lungs, alveolar lavage fluids were collected, and the number of inflammatory cells and the inflammatory factor levels were detected by using a Giemsa staining method and an ELISA method respectively. The right lungs were weighed, and lung indexes of the mice were calculated. The inferior lobes of the right lungs were taken, paraffin sections of lung tissue of the mice were manufactured according to Experimental Example 3, and pathologic changes of the lung tissue of the mice were observed and photographed. The remaining lung tissue was preserved at −80° C., and the levels of MMP-9 and NE were detected by using an ELISA method.

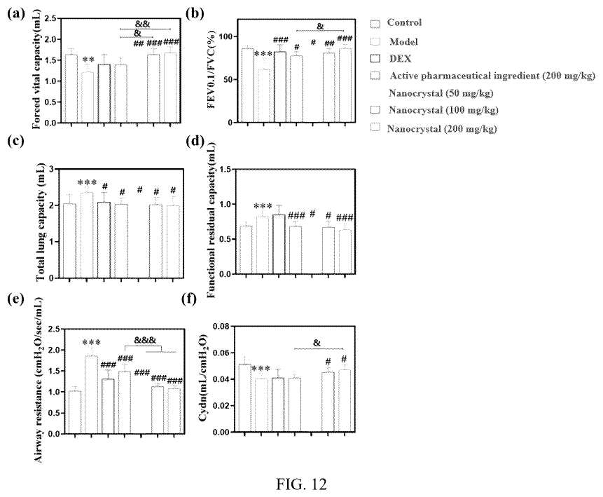

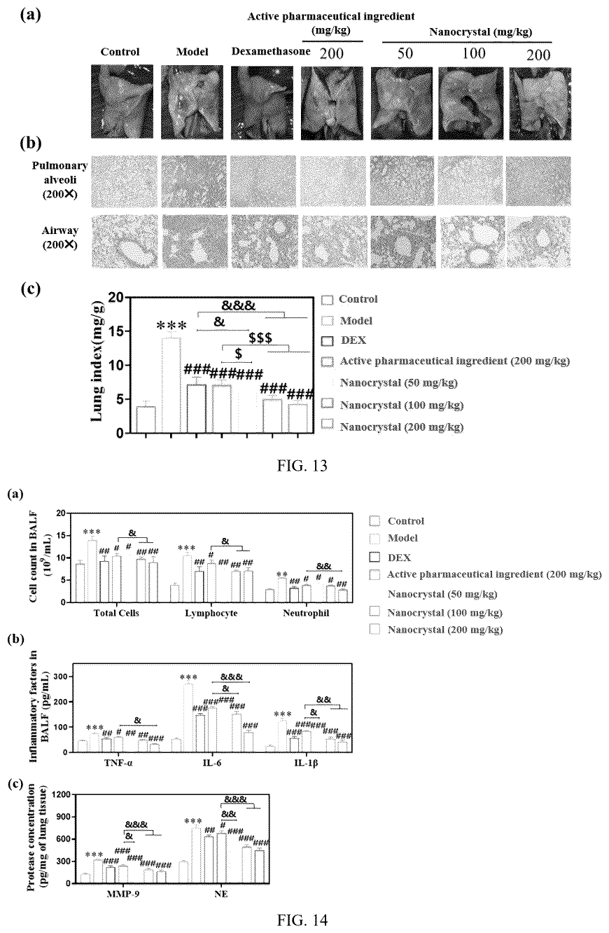

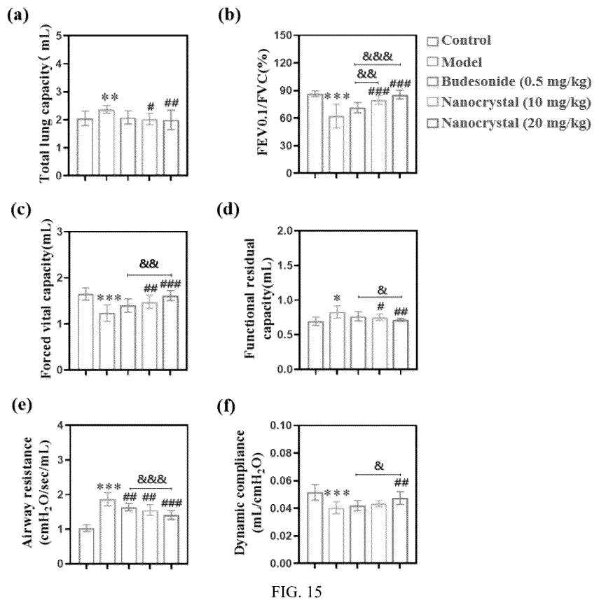

Results show that, the nanocrystals (100, 200 mg/kg) significantly increase FVC, FEV_{0.1}/FVC and Cydn, and reduce TLC, FRC and RI, thus improving a ventilation function of the chronic obstructive pulmonary disease mice (see FIG. 12 for the results). Except for Cydn, the 50 mg/kg nanocrystal may significantly improve the above indicators, and the effect is equivalent to that of the active pharmaceutical ingredient (200 mg/kg) (see FIG. 12 for the results). As for the effects on FVC, FEV_{0.1}/FVC, Cydn and RI, the effect of the nanocrystals of the same dosage is better than that of the active pharmaceutical ingredient. The nanocrystals (50, 100, 200 mg/kg) significantly reduce the lung index of the chronic obstructive pulmonary disease mice, and the effects of the various dosages of nanocrystals are significantly better than those of the active pharmaceutical ingredient; and lung injury symptoms such as alveolar wall destruction and fusion, bronchial wall thickening, and inflammatory cell infiltration in the chronic obstructive pulmonary disease mice are significantly alleviated (see FIG. 13 for the results). The nanocrystals (50, 100, 200 mg/kg) significantly inhibit the pulmonary inflammatory response of the chronic obstructive pulmonary disease mice, manifested by a significant decrease in total cell count, macrophage count, lymphocyte count, neutrophil count and levels of TNF-α, IL-6, and IL-1β in the mouse bronchial lavage fluids. The effect of the low-dosage nanocrystal is equivalent to that of the active pharmaceutical ingredient, and the effect of the nanocrystal of the same dosage is better than that of the active pharmaceutical ingredient (see FIG. 14a, b for the results). The nanocrystals (50, 100, 200 mg/kg) significantly inhibit the levels of MMP-9 and NE in the lung tissue of the chronic obstructive pulmonary disease mice, the effects of various dosages of nanocrystals are all significantly better than those of the active pharmaceutical ingredient (see FIG. 14c for the results), and the effect of the nanocrystals is equivalent to that of dexamethasone.

### Experimental Example 7: Efficacy Evaluation of Suspended Nebulization Inhalation Administration of Triacetyl Andrographolide Nanocrystal in Treatment of Chronic Obstructive Pulmonary Disease Mouse Models Constructed by Combination of Cigarette Smoking and LPS Instillation

The triacetyl andrographolide nanocrystal dry suspension obtained in Application Example 1 was used as a drug, and male KM mice were selected for experiments, 75 mice in total (20±2 g). Before the start of testing, the mice were randomly divided into 5 groups (n=15) by body weight, including a control group, a model group, a budesonide group (0.5 mg/kg), and low and high dosage nanocrystal administration groups (10 and 20 mg/kg by an effective content of triacetyl andrographolide).

The chronic obstructive pulmonary disease mouse models were constructed according to Experimental Example 5, and the mice were administrated via the lungs from day 92 to day 96, once every other day; and normal saline of the same volume was given to the mice in the control group and the model group. On day 97, mouse lung function indicators, lung indexes, alveolar lavage fluid inflammatory cell counts, detection of inflammatory factor and protease levels, and lung tissue section manufacturing and observation were all carried out according to Experimental Example 5.

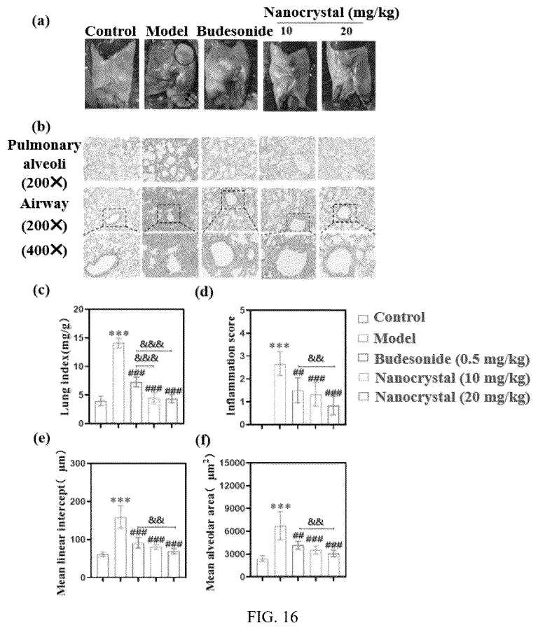

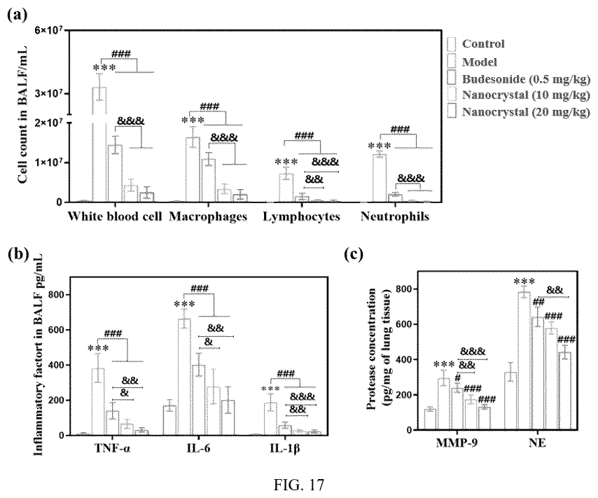

Results show that, the nanocrystal (20 mg/kg) significantly increases FVC, FEV_{0.1}/FVC and Cydn, and reduces TLC, FRC and RI, thus improving a ventilation function of the chronic obstructive pulmonary disease mice (see FIG. 15 for the results). Except for Cydn, the 10 mg/kg nanocrystal may improve the above indicators. The nanocrystals (10, 20 mg/kg) significantly reduce a lung index, an average alveolar intercept, an average alveolar area, and a pulmonary inflammation index in the chronic obstructive pulmonary disease mice (see FIG. 16 for the results). The nanocrystals (10, 20 mg/kg) significantly inhibit the pulmonary inflammatory response in the chronic obstructive pulmonary disease mice, manifested by a significant decrease in total cell count, macrophage count, lymphocyte count, neutrophil count, and levels of TNF-α, IL-6, and IL-1β in mouse alveolar lavage fluids (see FIGS. 17a, b for the results). Meanwhile, the nanocrystals (10, 20 mg/kg) significantly inhibit the levels of MMP-9 and NE in the lung tissue of the chronic obstructive pulmonary disease mice (see FIG. 17c for the results). The effect of the low-dosage nanocrystal is equivalent to that of budesonide, and except for TLC, the effect of the high-dosage nanocrystal for the above indicators is better than that of budesonide.

The triacetyl andrographolide nanocrystal or the dry suspension adopted in Experimental Examples 1-7 is replaced with the triacetyl andrographolide nanocrystal or the dry suspension thereof prepared in Examples 2-6, corresponding testing is performed under the same conditions in Experimental Examples 1-7, and results similar to those in Experimental Examples 1-7 are obtained.

The above is only preferred examples of the present disclosure and is not used to limit the present disclosure, and any modification, equivalent replacement, improvement, etc. made within the spirit and principle of the present disclosure should be included in the scope of protection of the present disclosure.

1. A triacetyl andrographolide nanocrystal, wherein the triacetyl andrographolide nanocrystal is mainly composed of triacetyl andrographolide, a stabilizer and an excipient, and an average particle size of drug particles in a triacetyl andrographolide nanocrystal suspension obtained by redissolving the triacetyl andrographolide nanocrystal in water, is less than 500 nm, and a PDI is less than 0.2; the triacetyl andrographolide nanocrystal suspension is prepared by adopting a high-speed shear anti-solvent method in combination with a high-pressure homogenization method, the nanocrystal is prepared further through spray-drying; the stabilizer is one or a mixture of soybean phospholipids and Poloxamer 188, or a mixture of soybean phospholipids and Tween 80; and the excipient is one or a mixture of the lactose and the mannitol.

2. The triacetyl andrographolide nanocrystal according to claim 1, wherein the triacetyl andrographolide nanocrystal has the same crystal form as a triacetyl andrographolide active pharmaceutical ingredient, both of them exhibit obvious crystalline diffraction peaks in their XRD spectra at 2θ values of 11.71°, 11.83°, 12.38°, 12.50°, 13.46°, 14.37°, 19.63° and 20.48°.

3. A triacetyl andrographolide nanocrystal preparation, comprising the triacetyl andrographolide nanocrystal according to claim 1 and conventional pharmaceutic adjuvants in the art.

4. The triacetyl andrographolide nanocrystal preparation according to claim 3, wherein the triacetyl andrographolide nanocrystal preparation comprises a solid preparation, a liquid preparation and a semi-solid preparation, and administration routes comprise oral administration, pulmonary inhalation administration, injection administration and topical administration.

5. A triacetyl andrographolide nanocrystal preparation, comprising the triacetyl andrographolide nanocrystal according to claim 2 and conventional pharmaceutic adjuvants in the art.

6. The triacetyl andrographolide nanocrystal preparation according to claim 5, wherein the triacetyl andrographolide nanocrystal preparation comprises a solid preparation, a liquid preparation and a semi-solid preparation, and administration routes comprise oral administration, pulmonary inhalation administration, injection administration and topical administration.

7. A method for treating lung injury-related diseases of a subject, comprising administrating the triacetyl andrographolide nanocrystal according to claim 1 to the subject, wherein the lung injury-related disease is acute lung injury or chronic obstructive pulmonary disease.

8. A method for treating lung injury-related diseases of a subject, comprising administrating the triacetyl andrographolide nanocrystal according to claim 2 to the subject, wherein the lung injury-related disease is acute lung injury or chronic obstructive pulmonary disease.

9. A method for treating lung injury-related diseases of a subject, comprising administrating the triacetyl andrographolide nanocrystal preparation according to claim 3 to the subject, wherein the lung injury-related disease is acute lung injury or chronic obstructive pulmonary disease.

10. A method for treating lung injury-related diseases of a subject, comprising administrating the triacetyl andrographolide nanocrystal preparation according to claim 4 to the subject, wherein the lung injury-related disease is acute lung injury or chronic obstructive pulmonary disease.

11. A method for treating lung injury-related diseases of a subject, comprising administrating the triacetyl andrographolide nanocrystal preparation according to claim 5 to the subject, wherein the lung injury-related disease is acute lung injury or chronic obstructive pulmonary disease.

12. A method for treating lung injury-related diseases of a subject, comprising administrating the triacetyl andrographolide nanocrystal preparation according to claim 6 to the subject, wherein the lung injury-related disease is acute lung injury or chronic obstructive pulmonary disease.

13. A method for preparing the triacetyl andrographolide nanocrystal according to claim 1, comprising the following steps:
(1) dissolving a drug triacetyl andrographolide in an organic solvent to obtain a drug solution, and dissolving a stabilizer in water to obtain a stabilizer solution;
(2) adding the drug solution in the stabilizer solution and dispersing the mixture by high-speed shear dispersion to obtain a coarse drug suspension;
(3) subjecting the coarse drug suspension to high-pressure homogenization to obtain a nanocrystal suspension of the triacetyl andrographolide; (4) adding an excipient to the nanocrystal suspension and preparing the triacetyl andrographolide nanocrystal by spray-drying.

14. The method for preparing the triacetyl andrographolide nanocrystal according to claim 13, wherein the organic solvent in step (1) is selected from one of ethanol and acetone, or a mixture thereof.

15. The method for preparing the triacetyl andrographolide nanocrystal according to claim 14, wherein in step (1), a mass volume ratio of the drug triacetyl andrographolide to the organic solvent is (0.20 to 0.40 g): 1 mL;
in step (2), a volume ratio of the drug solution to the stabilizer solution is 1:20 to 1:50, a mass ratio of the triacetyl andrographolide to the stabilizer in the coarse drug suspension is 1:1 to 5:1, and high-speed shear conditions are a shear stirring speed of 5000 to 10000 rpm and a shear time of 5 to 10 min;
in step (3), during high-pressure homogenization, a homogenizing pressure is 10000 to 20000 psi, and a number of homogenization is 10 to 20 times;
in step (4), a mass volume ratio of the excipient to the nanocrystal suspension of the triacetyl andrographolide is (0.03 to 0.10 g): 1 mL.

16. The method for preparing the triacetyl andrographolide nanocrystal according to claim 15, wherein process parameters during spray-drying in step (4) are an inlet temperature of 60° C. to 70° C., an outlet temperature of 30° C. to 40° C., a spray pressure of 0.1 to 0.2 Mpa, a sample injection rate of 1.0 to 2.0 mL/min, and a drying air volume of 0.5 to 1.0 m³/min.

17. A method for preparing the triacetyl andrographolide nanocrystal according to claim 2, comprising the following steps:
(1) dissolving a drug triacetyl andrographolide in an organic solvent to obtain a drug solution, and dissolving a stabilizer in water to obtain a stabilizer solution;
(2) adding the drug solution in the stabilizer solution and dispersing the mixture by high-speed shear dispersion to obtain a coarse drug suspension;
(3) subjecting the coarse drug suspension to high-pressure homogenization to obtain a nanocrystal suspension of the triacetyl andrographolide;
(4) adding an excipient to the nanocrystal suspension and preparing the triacetyl andrographolide nanocrystal by spray-drying.

18. The method for preparing the triacetyl andrographolide nanocrystal according to claim 17, wherein the organic solvent in step (1) is selected from one of ethanol and acetone, or a mixture thereof.

19. The method for preparing the triacetyl andrographolide nanocrystal according to claim 18, wherein in step (1), a mass volume ratio of the drug triacetyl andrographolide to the organic solvent is (0.20 to 0.40 g): 1 mL;
in step (2), a volume ratio of the drug solution to the stabilizer solution is 1:20 to 1:50, a mass ratio of the triacetyl andrographolide to the stabilizer in the coarse drug suspension is 1:1 to 5:1, and high-speed shear conditions are a shear stirring speed of 5000 to 10000 rpm and a shear time of 5 to 10 min;
in step (3), during high-pressure homogenization, a homogenizing pressure is 10000 to 20000 psi, and a number of homogenization is 10 to 20 times;
in step (4), a mass volume ratio of the excipient to the nanocrystal suspension of the triacetyl andrographolide is (0.03 to 0.10 g): 1 mL.

20. The method for preparing the triacetyl andrographolide nanocrystal according to claim 19, wherein process parameters during spray-drying in step (4) are an inlet temperature of 60° C. to 70° C., an outlet temperature of 30° C. to 40° C., a spray pressure of 0.1 to 0.2 Mpa, a sample injection rate of 1.0 to 2.0 mL/min, and a drying air volume of 0.5 to 1.0 m³/min.
